(19) United States
(12) Patent Application Publication
(10) Pub. No.: US 2021/0396405 A1
(43) Pub. Date: Dec. 23, 2021

(54) CENTRALIZED AEROSOL SOURCE EXTRACTION AND FILTRATION SYSTEM

(71) Applicant: David A Crank, Todd, NC (US)

(72) Inventor: David A Crank, Todd, NC (US)

(21) Appl. No.: 16/907,014

(22) Filed: Jun. 19, 2020

(51) Int. Cl. F24F 3/16; B01D 46/00; B01D 53/86; B01D 46/10; B01J 35/00; B01J 21/06; A61L 9/01; A61L 9/12; A61L 9/20

(52) U.S. Cl. CPC F24F 3/1603; A61L 2209/212; B01D 46/0028; B01D 46/0036; B01D 46/0032; B01D 53/86; B01D 46/10; B01J 35/004; B01J 21/063; A61L 9/01; A61L 9/122; A61L 9/205; B01D 2267/40; B01D 2273/30; B01D 2279/65; B01D 2255/20707; B01D 2255/802; F24F 2003/1667; A61L 2209/14; A61L 2209/12; A61L 2209/22; B01D 46/0049

(57) ABSTRACT

A system for providing a centralized aerosol source extraction and filtration system is disclosed. The system includes an aerosol capture component coupled to a set of aerosol capture ductwork being located at or near a patient's mouth while treatment is being provided and is coupled to a set of aerosol capture ductwork, a filter and fan component coupled to the set of aerosol capture ductwork for generating the necessary pressure differential to draw the air about the patient's mouth into the aerosol capture component along with any airborne particulates, contaminants, and pathogens, and a recycled clean air component for returning recycled clean air back to patient areas and workspaces through a set of recycled clean air ductwork arranged to create positive and directional airflow throughout the office space. The filter and fan component has one or more filter layer types to remove particulates, contaminants, and pathogens from an air flow received from the set of aerosol capture ductwork.

### TECHNICAL FIELD

This application relates in general to a system for providing dental air recirculation, and more specifically, to a system for providing a centralized aerosol source extraction and filtration system.

### BACKGROUND

Dental offices generally face a problem in that significant quantities of potentially hazardous aerosolized particles are generated by procedures performed in patients' mouths. This cloud of aerosol, which may contain viruses, bacteria, fungi, blood, and sputum, is able to drift throughout the office and potentially transmit disease to others both inside and outside the patient treatment rooms. This cloud of aerosols has been detected many hours after its formation.

Source capture within dentistry is not entirely new in and of itself. However, existing systems utilize a local extraction and filtration device that is placed within each dental operatory that typically occupies floor space or is atypically mounted to the wall of the operatory, which is also plugged into the wall, generates motor noise in the operatory, requires maintenance and filter management, and exhausts the air into the space from which it is extracted, potentially creating cross currents of air that carry the contaminated aerosols away from the suction end of the device and distributing them throughout the clinic. Other systems may be configured to collect and exhaust the aerosolized material outside the building into the larger environment, also exhausting the heated, cooled, and conditioned air from the dental office, whether filtered or not, thus increasing energy costs for the clinic. None of these systems are able to create as comprehensive an aerosol source extraction and filtration solution for an office space that captures bioaerosol-contaminated air at its multiple sources, saves clinic space by having only the extraction arm located within the space without the filtration and motor unit, minimizes noise in the patient areas by placing the fan in a central location away from these areas, and creates desirable directional airflow within the clinic.

Therefore, a need exists for a centralized system having fume extraction arms in close proximity to the sources of bio-aerosol generation, i.e. the patients' mouths, to remove the potentially contaminated air, sending it through a section of contaminated ductwork, to an air filtration unit and fan before returning the filtered air to common areas designated as uncontaminated spaces, from where it will naturally flow back into the contaminated patient treatment areas. Thus, negative pressure is generated at the intake terminus of the system, whereas positive pressure is generated at filtered air returning registers within the uncontaminated spaces of a clinic, leading to the creation of a perpetual current of directional airflow through the clinic. Air intake registers in common areas such as waiting rooms, laboratory spaces, hallways, office areas, and other spaces may also be fed into the air intake portion of the system to filter possible sources of contamination located in these areas as well. With proper design the centralized aerosol source extraction and filtration system may, in most office layouts, be able to generate directional airflow throughout the office areas that may reduce or eliminate possible areas of stagnant and contaminated air. The present invention attempts to address the limitations found in existing systems using the principles of operation as disclosed herein.

### SUMMARY

In accordance with the present invention, the above and other problems are solved by providing a system for centralized aerosol source extraction and filtration according to the principles and example embodiments disclosed herein.

In one embodiment, the present invention is a system for providing a centralized aerosol source extraction and filtration system. The system includes one or more aerosol capture components located at or near a patient's mouth while treatment is being provided and is itself coupled to an aerosol capture duct, a filter component and fan being coupled to the aerosol capture duct for generating the necessary pressure differential to draw the air about the patient's mouth into the aerosol capture component along with any airborne particulates, contaminants, and pathogens, and a recycled clean air component for delivering purified air back to patient areas and workspaces through a set of clean air return ductwork. The system in its totality generates negative air pressure at the contaminated air intakes and positive pressure at the clean air return registers, which leads to the generation of directional airflow throughout these office spaces that is useful to prevent the escape of contaminated air to the rest of the office. In offices with actual doors to patient operatories or improvised doors, it is possible for this directional airflow to create negative pressure isolation within the doored rooms. The filter and fan component may be located inside one housing or two separate housings that are joined via ducting. The filtration component has one or more filter layer types for the purposes of capture, filtration, attenuation, destruction, and/or adsorption of particulates, pathogens, and gases from an air flow received from the aerosol capture component.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment for a system for providing a centralized aerosol source extraction and filtration system.

FIG. 2 illustrates an example embodiment of an extraction arm hood and aerosol collector for use in a dental personal protection device according to the present invention.

FIG. 3 illustrates an example of a dental aerosol collector on a movable and articulatable arm as the point of source capture for a system for providing centralized aerosol source extraction and filtration in a dental office according to the present invention.

FIG. 4 illustrates an example embodiment of a filter and fan component within a system for providing a centralized aerosol source extraction and filtration system according to the present invention.

FIG. 5 illustrates an example of a filtered airflow diagram for a dental office according to the present invention.

FIG. 6 illustrates another example of a filtered airflow diagram for a dental office according to the present invention.

FIG. 7 illustrates yet another example of a filtered airflow diagram for a dental office according to the present invention.

### DETAILED DESCRIPTION

This application relates in general a system and method for providing a personal dental protection system, and more specifically, to a system for providing a centralized aerosol source extraction and filtration system according to the present invention. Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specifications and claims are approximations that may vary depending upon the desired properties sought to be obtained by the present disclosure. At the very least, and not as an attempt to limit the application of the doctrine of equivalents to the scope of the claims, each numerical parameter should be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

The terms “dental care provider,” and “user” refer to an entity, e.g. a human, using the system for providing a centralized aerosol source extraction and filtration system including any filter elements associated with the invention. The term used herein refers to one or more users.

The term “invention” or “present invention” refers to the invention being applied for via the patent application with the title “A Centralized Aerosol Source Extraction And Filtration System.”

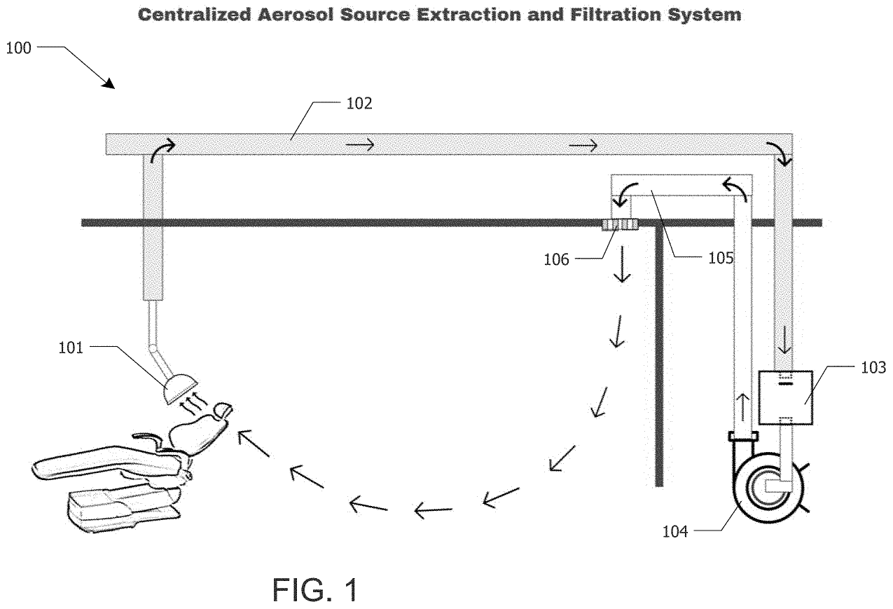

In general, the present disclosure relates a system and method for providing an office-wide personal dental protection device. FIG. 1 illustrates an example embodiment for a system for providing a centralized aerosol source extraction and filtration system. The aerosol source extraction and filtration system 100 comprises four basic components: an aerosol capture component 101 and 102, a filter component 103, a fan component 104, and a recycled clean air component 105 and 106. The set of aerosol capture components 101 is arranged to create negative and directional airflow throughout the office spaces. The aerosol capture component 101 is located within close proximity to a patient while treatment is being provided and is coupled to an aerosol capture duct 102 that is connected to the filter component 103. The aerosol capture component may also comprise a vent in the wall, ceiling or floor, a ceiling mounted hood similar in purpose to what is found over a stove, a hood such as a fume hood or biological safety cabinet that may be wall mounted, bench or cabinet mounted, or standing on the floor, and a hood over a patient.

The fan component 104 generates the necessary pressure differential to draw the air about the patient into the aerosol capture component 101 along with any airborne particulates, contaminants, and pathogens. The air and aerosol material is passed through a filter component 103 to remove from the airflow and/or neutralize any particulates, contaminants, or pathogens in the airflow before the fan component 104 passes purified air to the recycled clean air duct component 105 and into the office space by a fixed or adjustable vent component 106 located on the ceiling, wall, or floor.

The entire system is separate from an HVAC system while being a permanent built-in part of the office/clinic itself. The recycled clean air component 105 comprises a set of recycled clean air ductwork 105 coupled to the fan section 104 for transporting clean air back to patient areas and workspaces. The set of recycled clean air ductwork 105 is arranged to create positive and directional airflow throughout the office spaces. The location of recycled clean air return registers 106 coupled to the recycled clean air ductwork 105 are arranged to manage the airflow in a defined pattern. In some embodiments, the airflow through the recycled air ductwork 105 and return registers 106 may be dynamically controlled by the system to direct the purified air through portions of the office/clinic that are in use by patients and employees.

The system 100 may include additional components not shown herein. These additional components may include a dehumidifying and/or a humidifying element to further condition the recycled air depending upon the locale and time of year the system 100 is in use. The ductwork disclosed herein may be rigid or flexible, hidden above the ceiling or in walls, below the floors or be exposed as best fits the existing space used for the facility. Similarly, all recycled clean air return registers may be mounted on the ceiling, walls, floors, or exposed ductwork as needed.

The system 100 disclosed herein is described as it may be used within a dental office and dental clinic. One of ordinary skill in the art will recognize that the system 100 as recited in the attached claims may be used within many medical settings that may benefit from such an aerosol extraction process. By substituting a hood over the patient chair for the extraction arm or substituting ceiling intakes for the extraction arm, the system 100 may convert various treatment spaces, including an entire ward of a hospital, into an airborne infectious isolation ward in a very simple and cost effective way by creating negative pressure isolation with the system 100 as it is configured. System 100 may also be applicable to make the air in waiting/reception areas safer to breathe for many medical facilities. Other systems may utilize ceiling intakes as opposed to hoods or extraction arms, but the similar system configuration may provide a very high level of air filtration that simple modular units would not be able to provide. The use of the system 100 is therefore not intended to be limited to any one type of facility except as recited within the attached claims.

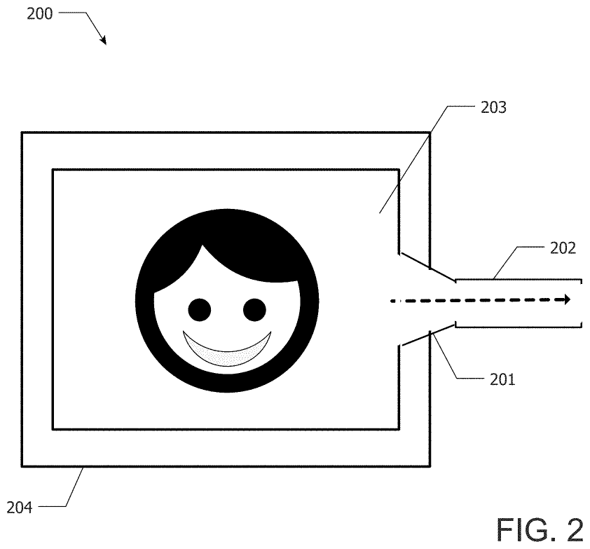

FIG. 2 illustrates an example embodiment of a dental office hood and aerosol collector for use as a dental personal protection device according to the present invention. In this embodiment, a dental hood 200 is placed anterior to the patient's face and mouth. The dental hood 200 contains a transparent center portion 203 that is surrounded by a transparent collection frame 204. The collection frame 204 may extend downward creating a workspace beneath the transparent center portion 203 near the patient. The collection frame 204 is shaped and arranged to permit a dental care provider to reach under the dental hood 200 to perform a procedure while viewing the patient through the transparent center portion 203. The dental hood 200, therefore, acts not only as an aerosol collection frame but also as a shield between the dental provider and the patient.

On one side of the transparent center portion 203, an extraction port 201 is located within the collection frame 204. The extraction port 201 is coupled to an exhaust line 202 that leads away from the dental hood 200. The exhaust line 202 is coupled to the aerosol capture ductwork 102 that is connected to the filter component 103 and fan component 104. The fan component 104 generates a pressure differential between the room air and the space below the dental hood 200 such that potentially draws all of the air and aerosol present about a patient's mouth into the extraction port 201 towards the filter component 103.

The size and shape of the dental hood 200 typically is selected by dental personnel. The size and shape of the dental hood 200 may vary depending upon the age, size, and needs of particular patients and the preferences of dental personnel. The dental hood 200 provides a benefit from the extraction of aerosol particulates from about a patient's mouth. The use of a transparent dental hood 200 provides a physical barrier between the patient and the dental care provider to minimize or eliminate the ability for splatter and debris to make contact with the dental providers. The only contact, therefore, between the patient and provider are the dental provider's hands that perform a procedure. Use of gloves, hand washing, and related hygiene of the dental provider's hands addresses any contact that may occur during a dental procedure. Although beneficial, use of a dental hood is not necessary for the system to function as an aerosol capture duct 102 having little or no extra collection surface as part of the present invention. The type of aerosol collection component 101 is not intended to require any particular type of hood or similar device and is not intended to be limited except as recited in the attached claims.

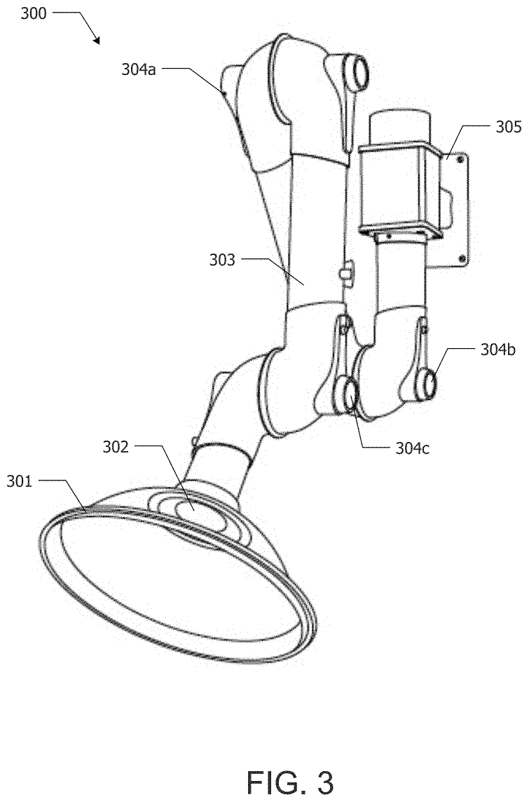

FIG. 3 illustrates an example of a dental aerosol collector on a movable and articulatable arm as the point of source capture for a system for providing a centralized aerosol source extraction and filtration system in a dental office according to the present invention. The dental hood 200 of FIG. 2 and 301 of FIG. 3 may be part of a retractable collection component 300. The hood 301 shown in FIG. 3 also contains an extraction port 302 that is coupled to an exhaust line 303 that is integrated into the movable and articulatable arm of the retractable collection component 300. The movable and articulatable arm may possess a plurality of movable joints 304a-c that may be arranged to place the hood collection device 301 at a desired Location near the patient. Other embodiments of the arm include, but are not limited to, non-jointed flexible arms with gooseneck-like bend-ability, telescopic arms, arms with internal or external articulating skeletal supports, and rigid arms of any type that permits an aerosol collection component to be moved into a desired orientation for use while treating a patient.

The exhaust line 303 provides a direct connection to the aerosol capture ductwork 102 which connects to the filter component 103. The exhaust line 303 passes through the plurality of movable joints 304a-c on the way from the extraction port 302 to the aerosol capture ductwork 102. One of ordinary skill will recognize that other retraction mechanisms through which the exhaust line 303 may pass may be used as the invention should only be limited by the limitations recited within the attached claims.

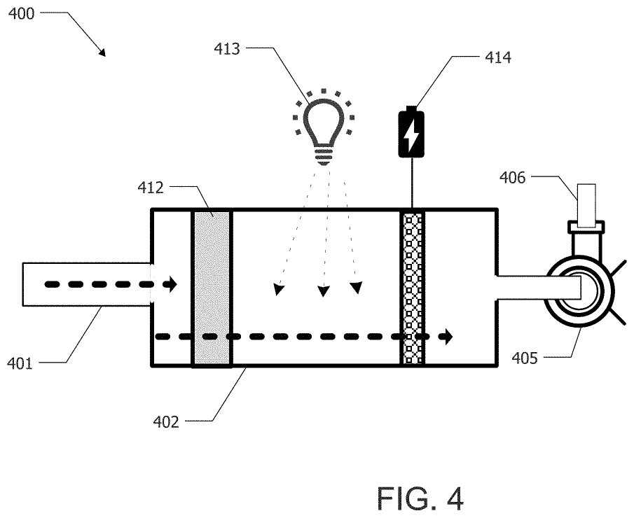

FIG. 4 illustrates an example embodiment of a fan and filter component within a system for providing a centralized aerosol source extraction and filtration system according to the present invention. The filter and fan component 400 comprises an aerosol capture intake 401, a filter component 402, a fan component 405, and a recycled clean air duct 406. The fan component 403 generates the necessary pressure differential to draw the air about the patient's mouth into the aerosol capture component 101 along with any airborne particulates, contaminants, and pathogens. The patient airflow passes through the filter component 402 on its way to the fan component.

The filter component 402 comprises a central filtration housing that contains one or more layers or types of air filtration 412-414. The particular filter elements within the filter component 402 comprises one or more of the following particulate filter 411 types that provide at or near HEPA filtering. The filter types may comprise: a Low Minimum Efficiency Reporting Value (MERV) filter, a chemical media filter such as activated carbon, a high MERV filter, a high efficiency particulate air (HEPA) filter, and/or an Ultra-Low Particulate Air (ULPA) filter. The plurality of filter elements may or may not contain additional disinfectant technology or materials including and not limited to electrostatic filter 413 and an ultraviolet germicidal irradiation (UVGI) lighting filter 412. Many other types of pathogen processing technologies, such as electrostatic filters, ionizing filters, UV lights, ozone plates, ozone generation, catalytic oxidation, such as TiO₂ photocatalytic filter, cold catalyst, molecular sieve, and diffusion filter, may also be included within one or more filter element types. A particular combination and arrangement of filter types may vary as desired to address a particular set of particulates and pathogens as determined by the dental professionals.

The filter and fan components 400 further comprises a central fan 406 or blower. In a preferred embodiment, the central fan 406 may be an air movement device capable of moving a sufficient quantity of air to create a pressure differential that extracts the aerosol components from about the patient. The central fan or blower 406 is recommended to be mounted after the filtration section 402; however, the central fan 406 may possibly be positioned before the filtration component 402 or within the filtration housing. The fan speed in a preferred embodiment should be established to maintain a bare minimum of 200 feet per minute for the filtered airflow. The use of various filter section layer types 412-414 may depend upon many different factors to capture, filter, attenuate, destroy, and/or adsorb particulates, pathogens, and gases. These factors may include the speed of the airflow through the filter component 402 as the effectiveness of a particular filter layer type may be affected by higher rates of airflow, the size and nature of the particulate and pathogen being addressed, and similar factors needed by a particular dental facility or clinic.

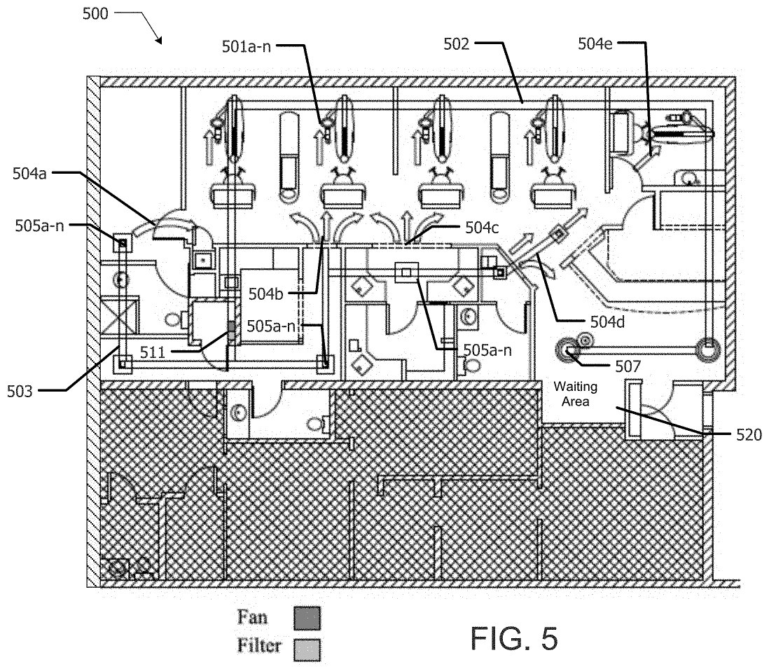

FIG. 5 illustrates an example of a filtered airflow diagram for a dental office according to the present invention. The dental office or clinic 500 has a plurality of dental operatory stations 501a-n. The plurality of dental operatory stations 501a-n each have an attachment to an aerosol capture duct 502 connected to a central aerosol capture ductwork 502. The central aerosol capture ductwork 502 removes aerosol particulates and pathogens via an aerosol capture component 101 from each operatory station 501a-n.

The central aerosol capture ductwork 502 is also coupled to one or more air intake vents 507 located within common areas. The central aerosol capture ductwork 502 terminates at a filter and fan component 511. The filter and fan component 511 generates return airflow 504a-e using a set of recycled clean air ductwork 503 having a set of return registers 505a-n. The set of recycled clean air 503 and the set of return registers 505a-n may be dynamically changed, either manually or electronically to change the return airflow 504a-e as desired.

The placement and angulation of the return air registers 505a-n determine the effectiveness of directional airflow achieved (vs. turbulence created), the thoroughness of office air circulation (bypassing entire portions of office air is possible if care is not taken), and is significant for balancing opposing air intake currents to prevent the mixing of contaminated with non-contaminated air.

In the embodiment of FIG. 5, the return airflow 504a-e is released within office space and common spaces away from the plurality of dental operatory stations 501a-n where the return airflow passes through doorways, around doors, around walls, and between other structural components of the office or clinic to create a directed airflow towards the dental operatory stations 501a-n and waiting area 520.

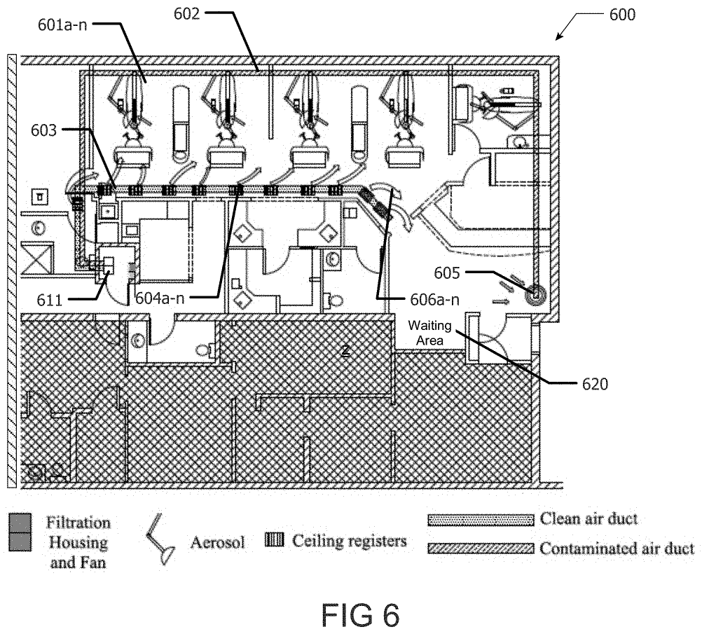

FIG. 6 illustrates another example of a filtered airflow diagram for a dental office according to the present invention. The dental office or clinic 600 has a plurality of dental operatory stations 601a-n. The plurality of dental operatory stations 601a-n each have an attachment to a central aerosol capture ductwork 602. The central aerosol capture ductwork 602 removes aerosol particulates and pathogens via the aerosol capture component 101 located within each dental operatory station 601a-n.

The central aerosol capture ductwork 602 is also coupled to one or more air input vents 605 located within common areas. The central aerosol capture ductwork 602 terminates at a filter and fan component 611. The filter and fan component 611 generates return airflow 606a-n through the set of recycled clean air ducts component 603 having a set of ceiling registers 604a-n. The set of recycled clean air ducts 603 and the set of ceiling registers 604a-n may be dynamically changed, either manually or electronically to change the return airflow desired.

Once again, the placement and angulation of the ceiling air registers 604a-n determines the effectiveness of directional airflow achieved, the thoroughness of office air circulation, and is significant for balancing opposing air intake currents to prevent the mixing of contaminated with non-contaminated air.

In the embodiment of FIG. 6, the return airflow is released within hallway and adjacent spaces adjoining to dental operatory stations 601a-n workspace where the ceiling registers 604a-n in the office or clinic create a directed airflow that circulates the hallway air into the dental operatory stations 601a-n and waiting area 620.

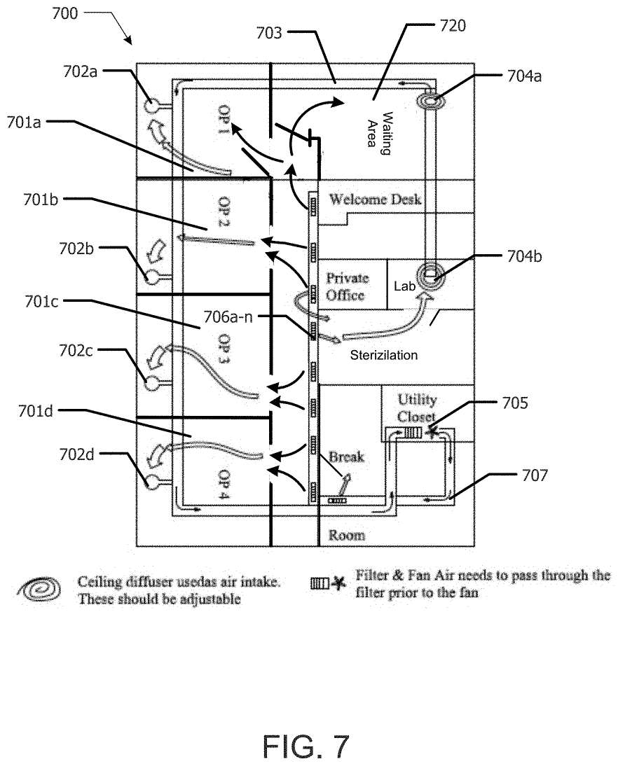

FIG. 7 illustrates yet another example of an airflow diagram for a dental office according to the present invention. The dental office or laboratory 700 has a plurality of dental operating locations 701a-d. The plurality of dental operating locations 701a-d each have an aerosol capture ductwork intake 702a-d connected to a central aerosol capture ductwork 703. The central aerosol capture ductwork 703 removes aerosol particulates and pathogens via the aerosol capture component 101 located within each dental operating location 701a-d.

The central aerosol capture ductwork 703 is also coupled to one or more air input vents 704a-b located within common areas. The central aerosol capture ductwork 703 terminates at a filter and fan component 705. The filter and fan component 705 generates return airflow using a set of recycled clean air ductwork 707 having a set of ceiling registers 706a-n. The set of recycled clean air ductwork 707 and the set of ceiling registers 706a-n may be dynamically changed, either manually or electronically to change the return airflow as desired.

As discussed above, the placement and angulation of the ceiling air registers 706a-n determines the effectiveness of directional airflow achieved, the thoroughness of office air circulation, and is significant for balancing opposing air intake currents to prevent the escape of contaminated air into spaces with non-contaminated air.

In the embodiment of FIG. 7, the return airflow is released within workspace adjacent to the dental operatory stations 701a-d by ceiling registers 706a-n in the office or clinic which create a directional airflow towards the dental operatory stations 701a-n and the waiting area 720.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. A system for providing a centralized aerosol source extraction and filtration system, the article comprises:
an aerosol capture component coupled to a set of aerosol capture ductwork;
a filter component coupled to the set of aerosol capture ductwork and recycled clean air ductwork for the purposes of capture, filtration, attenuation, destruction, and/or adsorption of particulates, pathogens, and gases from an air flow received from the aerosol capture component;
a fan component coupled to the set of aerosol capture ductwork or recycled clean air ductwork for generating the necessary pressure differential to draw the air about the patient into the aerosol capture components and air located within other designated areas of air contamination along with any airborne particulates, contaminants, and pathogens; and
a recycled clean air component for returning purified air back to patient areas and workspaces through a set of recycled clean air ductwork arranged to create beneficial directional airflow through the negative pressure generated at the intake terminus of the system and the positive pressure generated at filtered air returning registers located within the uncontaminated spaces of a facility.

2. The system according to claim 1, wherein the aerosol capture component is mounted on a movable arm permitting the aerosol capture component to be oriented about a patient's mouth while a dental procedure occurs.

3. The system according to claim 1, wherein the aerosol capture component is mounted above a patient permitting the aerosol capture component to be within close proximity while a medical procedure occurs.

4. The system according to claim 2, wherein the aerosol capture component comprises:
a dental hood having a transparent center portion that is surrounded by a collection frame; and
an extraction port coupled to an exhaust line that leads away from the aerosol capture component;
wherein the extraction port is coupled to a first side of the transparent center portion through the collection frame.

5. The system according to claim 2, wherein the aerosol capture component comprises a collection bowl having an extraction port coupled to an exhaust line that leads away from the aerosol capture component.

6. The system according to claim 1, wherein the filter and fan component comprises one or more filter layer types to capture, filter, attenuate, destroy, and/or adsorb particulates, pathogens, and gases from an air flow received from the set of aerosol capture ductwork.

7. The system according to claim 6, wherein one or more filter layer types may comprise: a low Minimum Efficiency Reporting Value (MERV) filter, a gas phase filtration media, a high MERV filter, a high efficiency particulate air (HEPA) filter, and/or an ultra-low particulate air (ULPA) filter.

8. The system according to claim 6, wherein one or more filter layer types further comprise filter layer types containing additional disinfectant technology and materials.

9. The system according to claim 8, wherein the additional disinfectant technology and materials comprise one or more of the following: an electrostatic filters, ionizing filters, UV lights, ozone plates, ozone generation, catalytic oxidation, such as TiO₂ photocatalytic filter, cold catalyst, molecular sieve, diffusion filter.

10. The system according to claim 1, wherein the recycled clean air component comprises:
a set of recycled clean air ductwork being coupled to the filter component for returning recycled clean air back to patient areas and workspaces and arranged to create positive and directional airflow throughout the office space; and
a plurality of return registers coupled to the recycled clean air ductwork and being arranged to manage the positive and directional airflow in a defined pattern.

11. A system for providing a centralized aerosol source extraction and filtration system, the article comprises:
an aerosol capture component coupled to a set of aerosol capture ductwork, the aerosol capture component is mounted on a fixed or movable arm permitting the aerosol capture component to be oriented about a patient's mouth while a dental procedure occurs comprising:
a dental hood having a transparent center portion that is surrounded by a collection frame; and
an extraction port coupled to an exhaust line that leads away from the aerosol capture component, the extraction port is coupled to a first side of the transparent center portion through the collection frame;
a filter and fan component coupled to the set of aerosol capture ductwork for generating the necessary pressure differential to draw the air about the patient's mouth into the aerosol capture component along with any airborne particulates, contaminants, and pathogens, the filter and fan component comprises:
one or more filter layer types to remove particulates and pathogens from an air flow received from the set of aerosol capture ductwork; and
one or more filter layer types comprise: a Low Minimum Efficiency Reporting Value (MERV) filter, a chemical media filter such as activated carbon, a high MERV filter, and an Ultra-Low Particulate Air (ULPA) filter; and
a recycled clean air component for returning recycled clean air back to patient areas and workspaces through a set of recycled clean air ductwork and recycled clean air registers, generating positive and directional airflow throughout the office space.
